(19) United States
(12) Patent Application Publication
(10) Pub. No.: US 2016/0310596 A1
(43) Pub. Date: Oct. 27, 2016

(54) COMPOSITION COMPRISING FERRIC SACCHARATE AND HIGH CONCENTRATIONS OF MICROENCAPSULATED LC-PUFA WITH A REDUCED OFF TASTE

(71) Applicant: NESTEC S.A., Vevey (CH)

(72) Inventors: Elodie SOUSSAN, Bern (CH); Rafael BERROCAL, Saint-Legier (CH); Matthieu BEDARD, Thun (CH); Marcel BRAUN, Konolfingen (CH)

(21) Appl. No.: 15/107,541

(22) PCT Filed: Dec. 19, 2014
(86) PCT No.: PCT/EP2014/078909

§ 371 Date: Jun. 23, 2016

(30) Foreign Application Priority Data
Dec. 27, 2013 (EP) 13199681.1

(51) Int. Cl. A61K 47/12; A23L 33/165; A23L 33/19; A61K 47/22; A61K 31/202; A61K 47/42; A61K 9/107; A23L 33/12; A23L 33/125

(52) U.S. Cl. CPC A61K 47/12; A23L 33/12; A23L 33/165; A23L 33/19; A23L 33/125; A61K 31/202; A61K 47/42; A61K 9/107; A61K 47/22; A23V 2002/00

(57) ABSTRACT

The present invention is directed to compositions, preferably nutritional compositions, preferably for infant, children, maternal, ageing care, elderly or health care nutrition. The present invention is further directed to pharmaceutical and/or nutraceutical compositions. The inventive compositions described herein preferably have a neutral or acidic pH, preferably a pH ranging from about 3 to about 7.5, said compositions being fortified with a high concentration of ferric saccharate and a high concentration of microencapsulated long chain-polyunsaturated fatty acids (LC-PUFA), preferably microencapsulated in a glassy matrix of dairy proteins and glucose. Preferably the inventive composition also comprises an antioxidant which is a radical scavenger, preferably along with a non-sensitive oil, said oil most preferably comprising medium chain triglycerides. The present invention also describes a method for preparing such compositions and the use of such compositions, preferably in the treatment of diseases as defined herein.

### FIELD OF THE INVENTION

The present invention is directed to compositions, preferably nutritional compositions, preferably for infant, children, maternal, ageing care, elderly or health care nutrition. The present invention is further directed to pharmaceutical and/or nutraceutical compositions.

The inventive compositions described herein preferably have a neutral or acidic pH, preferably a pH ranging from about 3 to about 7.5, said compositions being fortified with a ferric saccharate and a high concentration of microencapsulated long chain-polyunsaturated fatty acids (LC-PUFA), preferably microencapsulated in a glassy matrix of dairy proteins and glucose. Preferably the inventive composition also comprises an antioxidant which is a radical scavenger, preferably along with a non-sensitive oil and more preferably along with a protein, said oil most preferably comprising medium chain triglycerides and said protein most preferably comprising whey.

Further inventive compositions comprise said microencapsulated LC-PUFA along with microencapsulated ferric saccharate, preferably wherein the latter is microencapsulated in an alginate matrix.

The inventive compositions of the present invention preferably have improved 30 sensory properties, most preferably have a reduced off-taste, even in the presence of high concentrations of ferric saccharate and microencapsulated LC-PUFA. Similar or even further improved properties are also exhibited for inventive compositions wherein the ferric saccharate is also microencapsulated. Additional ingredients, as defined herein may also be included.

The present invention also describes a method for preparing such compositions and the use of such compositions.

### BACKGROUND

LC-PUFAs are essential components of our diet and scientific evidence supports that specific LC-PUFAs (such as docosahexaenoic acid (DHA) 22:6n-3) are important for brain and retina development, heart health (eicosapentaenoic (EPA) acid 22:5n-3) and a number of other emerging health benefits.

However, due to the presence of numerous double bonds, LC-PUFAs oxidize in the presence of oxygen, especially in the presence of iron. Lipid oxidation influences the quality of food products through flavour and taste deterioration and reduction in nutritional value. Off-flavour and off-taste formation such as rancidity, fishiness, metallic, fried fat, etc, results mainly from the degradation of primary oxidation products, such as peroxides, which can readily isomerise and degrade to produce volatile compounds. The deterioration of sensory properties is a major cause of consumer complaints in the food industry. Furthermore, shelf-life can be significantly impaired upon lipid oxidation.

As a result of a growing interest for enrichment of food with LC-PUFAs, bringing significant nutritional benefits, a lot of work has been reported on the development of technologies able to reduce degradation & off-notes due to LC-PUFA oxidation, including the following approaches:

### Masking Agent and Flavour

Focus has been put on masking agent and flavour for avoiding the fishy off notes in food matrices. However, flavour & masking agents do not stabilize LC-PUFAs, consequently the resulting LC-PUFA oxidation leads to a reduction of the nutritional value.

### Process & Packaging

Appropriate process & packaging should also decrease the rate of oxidation of LC-PUFA in food matrices e.g. via a separation of LC-PUFAs or iron from the rest of the food matrix. However, this solution is really expensive and not applicable for every type of product.

### Ingredients to Stabilize LC-PUFA Such as Encapsulation Technologies or Specific Antioxidants

Some of these solutions are based on ingredients able to stabilize LC-PUFA such as encapsulation technologies or specific antioxidants. However, these solutions are preferably specific for the selected type of food matrices, and are tailored around one specific encapsulated ingredient only.

Accordingly, it is an object of the present invention to provide a composition comprising iron and high amounts of LC-PUFA but nevertheless said composition having a reduced off taste.

A further object of the present invention is to provide a composition with improved stability, preferably having an extended shelf life.

A yet further object of the present invention is to provide a composition which is able to stabilise LC-PUFA in a liquid or powder product.

A further object is to provide a composition comprising iron and a high amount of LC-PUFA, for use as a medicament, preferably for use in the treatment of diseases requiring a high amount of LC-PUFAs.

A yet further object is to provide a method for preventing or reducing the oxidation of LC-PUFA, said method preferably preventing or reducing the off-taste of LC-PUFA.

### DESCRIPTION OF THE INVENTION

As described herein, the object underlying the present invention is preferably to be accomplished by means of the independent claims as attached. The dependent claims advantageously illustrate further preferred aspects of the inventive embodiments. Likewise or even further preferred aspects are outlined in the description.

The composition of the present invention is based on a specific adjustment of ingredients, wherein the present inventors have surprisingly found that a combination of ferric saccharate and microencapsulated LC-PUFA in the inventive composition herein results in a composition with a significantly reduced off taste, even where ferric saccharate and high concentrations of microencapsulated LC-PUFA are contained therein. It is furthermore also advantageous that the present invention comprises ferric saccharate which is preferably microencapsulated.

The composition of the present invention permits to fortify liquid products and/or powder products (from neutral pH to acidic pH) both with LC-PUFA and iron, while keeping the off-notes resulting of LC-PUFA oxidation as low as possible. This was assessed by a trained professional panel after tasting different emulsion-based compositions at day+1. Particularly, tasting may be carried out in milk, milk products, such as yoghurt, and fruit juice products.

The term “fortification” as used herein means to supplement or add nutrients that may be lacking in the overall diet to the inventive composition or any composition in the art, preferably using the inventive process as described herein.

Hence, according to a first embodiment, the object of the present invention is solved by a composition fortified with ferric saccharate and a high concentration of microencapsulated LC-PUFA, said composition preferably having improved sensory properties.

In the context of the present invention a high concentration of LC-PUFA is preferably understood as an amount being typically from about 0.02 to 10% by weight of the composition, preferably from about 0.02 to 5% by weight of the composition, by weight of the composition.

According to one likewise preferred aspect the microencapsulated LC-PUFA is derived from fish oil and/or algae oil, preferably DHA or EPA, or is provided in form of fish oil and/or algae oil, preferably DHA or EPA, and most preferably is DHA and/or EPA.

The inventive composition as described herein preferably contains also at least one antioxidant which is radical scavenger, preferably tocopherol (vitamin E). More preferably said radical scavenger is present in about 0.001% to about 1% by weight of the composition.

In some aspects of the present invention, the microencapsulated LC-PUFA is microencapsulated in a glassy matrix of dairy proteins and glucose. In a preferred aspect the present invention comprises said microencapsulated LC-PUFA along with ferric saccharate, which may also be microencapsulated, preferably in an alginate matrix,

In case the LC-PUFA is microencapsulated in a glassy matrix of dairy proteins and glucose, such a glassy matrix of dairy proteins can be prepared from any dairy protein available and suitable for this purpose, e.g. whey protein, casein, caseinate, milk proteins, β-lactoglobulin, α-lactalbumin, etc. Encapsulation may be carried out using techniques known in the art. Preferably LC-PUFA is encapsulated in a glassy matrix of dairy proteins as described in WO 2011/008097 A1 of Friesland Brands B.V., NL or can be obtained from FrieslandCampina Kievit under the trade name NIF powder.

As defined herein, the inventive composition is also fortified with ferric saccharate, preferably a high concentration of ferric saccharate. The inventors have surprisingly found that the use of ferric saccharate as iron source in the inventive composition prevents the generation of off-notes that is normally caused by mixing LC-PUFA and iron. Ferric saccharate provides the best sensory properties and no significant off-notes when admixing same with microencapsulated LC-PUFA in an inventive composition, compared to similar compositions comprising another iron source, evidencing that no significant, preferably no oxidation of LC-PUFAs occur. According to one preferred aspect the ferric saccharate comprised in the inventive composition is microencapsulated in an alginate matrix. Such an alginate matrix may be any alginate matrix suitable for a skilled person to prepare a microcapsule comprising ferric saccharate. Methods for encapsulating are known to a skilled person. In the context of the present invention, ferric saccharate may be encapsulated in an alginate matrix as is described in WO 2010/040789 A1 of AB-Biotics. Alternatively, ferric saccharate encapsulated in an alginate matrix can be obtained e.g. from AB-Biotics under the name AB-FORTIS.

In the context of the present invention, ferric saccharate is contained in the inventive composition to preferably provide an amount of iron, preferably a high amount of iron, from about 0.0001% to about 1% by weight of the composition, preferably from about 0.001 to 1, more preferably from about 0.001 to 0.1% by weight of the composition.

According to one further preferred aspect the composition of the present invention comprises a protein source, the protein source preferably being contained in the composition as an additional ingredient to proteins used for encapsulation.

Hence, according to one preferred aspect, the composition of the present invention comprises a(n additional) protein source, typically selected from vegetable or animal sources, preferably from dairy sources. Furthermore, fractions or partial hydrolysates of proteins may be present in the solution, preferably of proteins as defined herein. Preferably, the inventive composition comprises as a(n additional) protein source dairy protein, more preferably whey protein, either alone or in combination with other proteins selected from vegetable, such as soy protein, or animal sources. Particularly preferable the protein source is a dairy protein, such as defined above, more preferably whey protein and/or caseinate, most preferably whey protein. For example the suitable whey protein source is selected from the group comprising or consisting of e.g. whey protein, whey protein isolate (WPI), acidified whey protein isolate, whey protein concentrate (WPC), cheese whey, more preferably WPCs having a protein/dry matter content of 30-89%, and whey protein isolates (WPI), having a protein/dry matter content of 90% or higher, whey powder and combinations thereof and the like either alone or in combination. Generally, the inventive composition may comprise proteins as defined above as native or denatured proteins, fractions and/or (partial) hydrolysates of such proteins or a mixture thereof.

For purposes of the present invention, the composition as defined herein preferably comprises about 0.1% to 25% protein by weight of the composition, preferably calculated on basis of the protein contained in the inventive composition additionally to the protein used for encapsulation, more preferably about 5 to 15% by weight protein calculated on basis of the protein contained in the inventive composition additionally to the protein used for encapsulation, which is preferably whey protein.

The amount of protein is typically further determined by the person skilled in the art based on the desired nutritional properties of the inventive composition. For example if the inventive composition is an infant formula, the inventive composition preferably comprises protein as defined herein which provides about 4 to about 30%, more preferable 8 to 20% of the total energy of the composition. In another example, if the inventive composition is growing-up milk the inventive composition preferably comprises protein as defined herein which provides about 11 to 18% of the total energy of the composition.

The inventors have surprisingly found that when whey protein is contained in the inventive composition as an ingredient additional to those proteins used for encapsulation this even further prevents or reduces the oxidation of LC-PUFA in a liquid or powder product, whey protein acting synergistically with the combination of microencapsulation of LC-PUFA and the use of ferric saccharate as iron source.

According to one preferred aspect, the composition of the present invention comprises a carbohydrate source. The carbohydrate source as contained in the inventive composition typically may be selected from any suitable carbohydrate source which preferably also functions as a sweetener. Preferably, the carbohydrate source employed in the inventive composition may preferably be selected from the group consisting of sucrose, preferably castor sugar, fructose, maltodextrin, fibers, corn syrup, high fructose corn syrup, corn starch, lactose, glucose, dextrose, maltose and combinations thereof, etc. and the like either alone or in combination.

Preferably the carbohydrate source provides 40 to 80% of the total energy of the inventive composition, although the amount of carbohydrate employed may vary depending on the product and on the nutritional needs of the consumer.

Sweeteners, either derived from carbohydrates or further sources, can optionally be used although this will depend on the product type.

According to one other preferred aspect, the composition of the present invention comprises a fat source. Particularly preferred, the composition comprises other kinds of fat in addition to the LC-PUFA. Such fat can be any fat suitable for the kind of product. It has however been discovered that stabilization, i.e. preventing or reducing the oxidation of LC-PUFA can further be improved in the presence of a non-sensitive oil, preferably in the form of saturated or mono-unsaturated fatty acids, such as for example medium chain triglycerides (MCT). A composition further comprising a source of saturated and/or mono-unsaturated fatty acids, preferably a source of MCT is therefore preferred for the purpose of the present invention.

Furthermore, according to one preferred aspect, the fat as contained in the inventive composition typically may be selected from any suitable fat source, selected from the group comprising or consisting of coconut oil, preferably fractionated coconut oil, lemon oil, dietary fats, vegetable oil, such as sunflower oil, preferably high oleic sunflower oil, canola oil, corn oil, soybean oil, sesame seed oil, safflower oil, walnut oil, evening primrose oil, peanut oil, cottonseed oil, rapeseed oil, olive oil, macadamia oil, palm oil, palm kernel oil, or mixtures thereof, etc., either alone or in combination. A fat which is a source of MCT is preferred for the purpose of the present invention.

According to the present invention, fats are preferably selected from (fat) molecules composed of individual carbon atoms linked into chains ranging from 2 to 24 carbon atoms in length.

For the purposes of the present invention, the composition as defined herein preferably comprises about 0.1% to about 90% fat by weight of the composition, preferably about 0.5 to about 10% by weight, or about 5 to about 15% by weight, or about 7 to about 30% by weight, or about 20 to about 60% by weight, or about 40 to about 80% by weight, or about 70 to about 85% by weight of the composition. In the above context the term “%” is preferably defined as “% by weight” and in this context reflects the total amount of the fat.

Preferably the fat provides 5-40% of the total energy of the inventive composition, although the amount of carbohydrate employed may vary depending on the product and on the nutritional needs of the consumer.

Medium Chain Triglycerides (MCTs) as may be used herein are typically composed of 6 to 10, or 6 to 11, or 6 to 12 carbon links. Because of their shorter chain length MCTs have a number of unique properties which give them advantages over the more common LCTs. The MCT's employed in the present invention are preferably sourced from fractionated coconut oil, macadamia oil, palm oil, palm kernel oil, milk fat, etc. and combinations thereof.

The oil as may be employed in the inventive composition is selected depending on the type of product. For instance, for milk based products milk fat will be preferred.

A non-sensitive oil such as MCT is preferably contained in the inventive composition as the inventors have surprisingly found that MCT prevents or further reduces oxidation of LC-PUFA and improves the sensory properties by reducing fishy and painty off-notes generated by oxidation, such non-sensitive fat acting synergistically with the combination of microencapsulation of LC-PUFA and the use of ferric saccharate as iron source. Hence MCT is preferably included in the inventive composition since this further prevents or reduces the off-taste of LC-PUFA.

Most preferably, the inventive composition contains at least one antioxidant which is radical scavenger, preferably tocopherol (vitamin E), as the inventors have surprisingly found that such an antioxidant prevents or further reduces oxidation of LC-PUFA and improves the sensory properties by reducing fishy and painty off-notes generated by oxidation, such antioxidant acting synergistically with the combination of microencapsulation of LC-PUFA and the use of ferric saccharate as iron source. Tocopherol (vitamin E) is further advantageous in that it also has a nutritional effect.

Most preferably, the antioxidant which is a radical scavenger is present in about 0.001% to about 1% by weight of the inventive composition. Preferably about 0.005 to about 0.2%, or about 0.1 to about 0.5%, or about 0.3 to about 0.8%, or about 0.6 to about 0.9% by weight of the composition.

Optionally, the inventive composition may also contain other ingredients, depending on the intended use of the composition. Such ingredients can be added for nutritional purpose, such as micronutrients. Alternatively they can be added for technical reasons, such as for example to improve product stability, as would be the case for example for emulsifiers or for hedonic purposes.

For the purposes of the inventive composition, emulsifiers may also be present, which can include, but are not limited to, proteins, protein hydrolysate. Emulsifiers may also be selected from food-grade emulsifiers like lecithin, mono- and di-glycerides, proteins and sugar esters, such as for example sorbitan esters which are commercially available for example under the name Tween from diverse suppliers.

Particularly preferred emulsifiers are mono- and/or diglycerides of fatty acids, preferably, stearyl mono- and/or diglycerides. Various sources of protein or protein hydrolysate may be employed; milk proteins such as whey protein and caseinate are preferred.

Emulsifiers further include modified starches, such as Hi cap. Such modified starches can, e.g. be modified by reaction with n-octenylsuccinyl anhydride (NOSA). Mono- and diglycerides, distilled mono-glycerides, egg yolk and lecithin, which may be present either alone or in combination. Said emulsifiers may be present in an amount between about 0.05 to about 1%, preferably about 0.1 to about 0.2%, or about 0.15 to about 0.3%, or about 0.18 to about 0.4%, or about 0.35 to about 0.5%, or about 0.45 to about 0.65%, or about 0.55 to about 0.8%, or about 0.7 to about 0.9% by weight of the inventive composition.

Acids may also be employed in the inventive composition, preferably citric acid and/or malic acid and/or ascorbic acid and/or lactic acid and/or succinic acid and/or acetic acid. The acid can be added in liquid or dry form, such as in hydrated or anhydrous form and the like. Preferably, the inventive composition has a neutral or acidic pH. The inventive composition, which is preferably a semi-liquid/semi-solid composition, most preferably a liquid composition, is preferably adjusted to a pH of about 3.5 to about 7.5, more preferably to a pH of about 4 to about 7.

When the product is in powder form and intended to be reconstituted in water, the 25 pH refers to the pH of the product once reconstituted with water.

Said inventive composition may also comprise micronutrients selected from vitamins, minerals and trace elements, which may be present either alone or in combination. Alternatively, for some embodiments the inventive compositions may also not contain any micronutrients. The term “micronutrient” as used herein refers to vitamins and (dietary) minerals that are required in the human diet in very small amounts. The amounts of specific vitamins and minerals in the inventive composition typically may be determined by one of skill in the art.

The term “vitamin” as used herein, refers to any of various organic substances essential in minute quantities to the nutrition of most animals act especially as coenzymes and precursors of coenzymes in the regulation of metabolic processes. Vitamins have diverse biochemical functions, including function as hormones (for example, vitamin D), antioxidants (for example, vitamin C and vitamin E), and mediators of cell signalling, regulation of cell growth, tissue growth and differentiation (for example, vitamin A). The B complex vitamins, which is the largest in number, function as precursors for enzyme cofactor biomolecules (co-enzymes) that help act as catalysts and substrates in metabolism. For instance Vitamin B₆ and Vitamin B₁₂. Other Vitamins which may be present include Vitamin K, Thiamin, Riboflavin, Niacin, Folic Acid, Biotin and Pantothenic Acid. Any of these vitamins may be incorporated, preferably Vitamin C and/or vitamin E.

Dietary minerals, as may also be contained in the inventive composition, are chemical elements other than carbon, hydrogen, nitrogen, and oxygen that are required to sustain the health of living organisms. In humans, dietary minerals can include calcium, magnesium, phosphorus, potassium, sodium, and sulphur. Preferably, calcium is contained in the inventive composition and optionally at least one further dietary mineral as described before.

Furthermore, minerals that are needed in relatively small quantities and may be referred to as trace elements, for example, chromium, cobalt, copper, chloride, fluorine, iodine, manganese, molybdenum, selenium, and zinc.

In some preferred embodiments of the inventive composition zinc is not included therein.

The inventive composition as described herein can include any combination of vitamins, minerals and trace elements as mentioned before that is useful in providing appropriate nutrition to the patient. The vitamins, minerals and trace elements may be used in the form of a mixture or formulation.

Optionally, the composition of the present invention may also contain as optional ingredients probiotics, prebiotics, minerals, thickeners, buffers or agents for pH adjustment, chelating agents, colorants, emulsifiers, excipients, flavor agents, osmotic agents, preservatives, stabilizers, sugar, sweeteners, texturizers, and/or vitamins. For example, the compositions may contain emulsifiers and stabilizers such as soy lecithin, citric acid esters of mono- and di-glycerides, and the like. These optional ingredients can be added in any suitable amount and type, preferably as defined herein.

In this context, probiotics are preferably to be understood as food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See Salminen S., et al., “Probiotics: how should they be defined?” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host.

Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

A prebiotic in the context of the present invention is preferably a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn Gibson et al., “Dietary Modulation of the Human Colonic Microbiota: Introducing the Concept of Prebiotics,” J. Nutr., 125: 1401-1412 (1995).

Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

In some embodiments the composition of the present invention advantageously further comprises a flavour ingredient, which is selected according to the product requirements. In some embodiments, flavour may also be employed as a masking agent to even further improve the sensory properties of the composition, if required, or may be used to impart the composition with a more appealing flavor, particularly in the field of nutrition to increase acceptance of the end user.

The inventive composition furthermore may comprise water. The amount of water present by weight of the inventive composition may vary according to the product type, preferably water is present in the inventive composition in about 2% to about 95% by weight, or about 3 to about 10%, or about 5 to about 13%, or about 11 to about 20%, or about 15 to about 40%, or about 30 to about 50%, or about 45 to about 65%, or about 60 to about 80%, or about 70 to about 95% by weight of the inventive composition. The amount of water can also be much lower, for example when the composition is in the form of a powder.

The compositions as described herein are either solids (such as for example powders), liquids or semi liquids/semi solids, although liquids are particularly preferred. e.g. an oil-in-water emulsion.

In a most preferred aspect the inventive composition is able to stabilise LC-PUFA in a liquid or powder product. Stabilization of LC-PUFA in a liquid or powder product preferably means that oxidation of LC-PUFA is prevented or at least significantly reduced, such as to provide no or at least a diminished off-flavour caused by the oxidation products of LC-PUFA when compared to a composition comprising the same ingredients but either other LC-PUFA and/or another kind of iron source. Such an off-flavour can be tested and verified by a skilled person following accepted standards of sensory testing, such as for example the preference test.

According to a particularly preferred embodiment, the object underlying of the present invention is preferably solved by a composition, which typically comprises:

The LC-PUFA, preferably derived from fish oil or algae oil, most preferably derived from fish oil, is preferably encapsulated in a glassy matrix of dairy proteins and glucose. Said LC-PUFA preferably comprises from 20 mg to 3 g DHA and/or EPA per 100 g product typically along with encapsulated ferric saccharate, preferably ferric saccharate encapsulated in alginate matrix. The ferric saccharate is preferably present from 0.1 mg to 1 g per 100 g of product. Said LC-PUFA and ferric saccharate are preferably provided in the composition along with saturated or monounsaturated fat/oil, preferably comprising medium chain triglycerides in an amount of 0.1 g to 90 g per 100 g product along with a radical-scavenger antioxidant, preferably vitamin E in an amount of 0.001 g to 1 g per 100 g product, preferably along with whey protein in an amount of 0.1 g to 20 g per 100 g product.

Preferably the composition of the present invention as defined herein may be a nutritional composition, a pharmaceutical composition and/or a nutraceutical product. According to a preferred aspect the composition of the present invention is a nutritional composition.

Furthermore, the composition of the present invention may be for use as a supplement or may be used as a sole source of nutrition, e.g. as a full meal. The inventive composition may be furthermore suitable for use or used in the treatment of a disease as defined herein, particularly for use as a medicament. Additionally, the inventive composition may preferably be packaged, preferably providing a unit or dose for administration.

A nutritional composition as defined herein is preferably designed and used for infant, children or adult. For infant nutrition, the nutritional composition is preferably an infant formula. For adult nutrition, it is preferably used as a maternal nutrition or as an ageing care nutrition product. Alternatively the inventive nutritional composition can be a health-care product, preferably for health-care nutrition, i.e. a product intended for specific nutrition of sick people, preferably of sick people suffering from a disease or disorder as defined herein.

In the context of the present invention, an infant is preferably defined herein as being up to 2 years of age, whereas children are defined as being from 2 to 7 years of age.

Also in the context of the present invention maternal nutrition is preferably defined as being for pregnant and lactating women, and furthermore encompasses preconception administration to a woman willing to have a baby.

Also in the context of the present invention, an ageing care nutrition is preferably defined as being a product intended for elderly people or for adults willing to reduce the adverse effects of ageing.

Preferably, the nutritional composition is a food matrix, a beverage or a food supplement. For instance, for children as defined herein, the present invention can be utilized in the form of growing-up milk. The term “food supplement” as used herein refers to an inventive composition that may be added to the diet or a meal thereof.

A nutritional composition is typically defined herein as being any type of food in liquid or powder form, wherein in this context said nutritional composition typically contains proteins and/or fat and/or carbohydrate. Proteins, fat and carbohydrates as well as any further ingredients are preferably as defined above and may be selected as mentioned in the context of the inventive composition.

Within the context of the present invention, proteins, fats and carbohydrates are typically the nutritional ingredients of the inventive nutritional composition, which are selected depending on the product type. However, whenever acceptable for the product type, the saturated or monounsaturated fat/oil, preferably MCT shall be selected as fat. Also, whenever possible for the product type, it is preferred to use whey, at least for part of the protein, if possible, preferably only whey.

Preferably, the composition of the present invention could be obtained by any process suitable for a skilled person. More preferably, the inventive composition may be obtained or is obtainable by a method for preparing a composition as defined herein.

According to a further embodiment, the object underlying the present invention is therefore preferably also solved by a process for preparing a composition, preferably a composition as defined herein. The present invention hence describes a composition as described above, preferably a composition obtained or obtainable according to a process for preparing such a composition as defined herein. In this regard, said process may contain or comprise any of the amounts and ingredients as defined for the inventive composition.

According to a particularly preferred aspect the object underlying the present invention is solved by a process for the preparation of a composition, more preferably a composition as described herein, which comprises the step of mixing or dry blending the ingredients as defined herein above to obtain a mixture, which is most preferably the inventive composition as described herein.

Said process, preferably further comprising the steps of
(a) carrying out at least one heat treatment step of said mixture obtained after the mixing or dry blending the ingredients; and
(b) preferably homogenizing the mixture before or after the heat treatment step.

Said process is preferably carried out at a pH between about 3.5 and about 7.5 or the mixture is adapted accordingly, preferably to a pH as defined herein above for the inventive composition.

If acids are employed in said process, the acid(s) can be added in liquid or dry form, such as in hydrated or anhydrous form and the like. Acids are preferably as defined above.

Advantageously, said process includes steps such as heat treatment and homogenization which result in improved safety and quality of the product. In the compositions of the present invention, LC-PUFA is advantageously stabilized in such a way that oxidation is prevented even when the relatively aggressive process steps of heat treatment and homogenization are carried out. Therefore, the composition of the present invention retains good sensory properties, as a consequence of limited oxidation of LC-PUFA during heat treatment and homogenization.

The inventive process preferably results in a solid, liquid or semi-liquid/semi-solid composition, most preferably a liquid composition. Hence, the inventive composition may also be present as a solid composition, e.g. a powder, or a liquid or semi-liquid/semi-solid composition.

When the inventive composition is in solid form, such as a powder, the process should preferably include the steps of spray-drying, freeze drying or fluid bed agglomeration.

According to a further embodiment, the object underlying the present invention is preferably also solved by a method for preventing or reducing the oxidation of LC-PUFA in a composition comprising LC-PUFA and iron, comprising adding LC-PUFA to the composition in microencapsulated form and adding iron in the form of ferric saccharate, wherein preferably the LC-PUFA is microencapsulated in a glassy matrix of dairy proteins and glucose. The composition, preferably the resulting composition, comprising LC-PUFA and iron is preferably as defined herein.

Preventing or reducing the oxidation of LC-PUFA is as already defined herein above for the inventive composition.

According to a yet further embodiment, the object underlying the present invention is preferably also solved by a method for preventing or reducing the off-taste of LC-PUFA in a composition comprising LC-PUFA and iron, comprising adding LC-PUFA to the composition in microencapsulated form and adding iron in the form of ferric saccharate, wherein preferably the LC-PUFA is microencapsulated in a glassy matrix of dairy proteins and glucose. Most preferably the ingredients of such composition are as defined above for the inventive composition. The resulting composition, comprising LC-PUFA and iron is preferably as defined herein.

Preventing or reducing off-taste of LC-PUFA is defined as preventing or reducing the off-taste, such as rancidity, fishiness, metallic, painty, fried fat, etc., when compared to a composition comprising the same ingredients but either other LC-PUFA and/or another kind of iron source. Such an off-flavour can be tested and verified by a skilled person following accepted standards of sensory testing, such as the preference test.

According to a further embodiment, uses of the inventive compositions as described herein, either as described initially or as obtained or obtainable according to the inventive process, are contemplated. The inventive composition is particularly suitable for the use in the dietary management of diseases or disorders as defined herein or for the fortification of food.

The term “fortification” as used herein also includes addition of nutrients to a food or nutritional composition, preferably during the inventive process as described herein that may be lacking in the overall diet. Such nutrients include, but are not limited to folate, vitamins A and D, preferably vitamin C as an oxygen-scavenger, calcium, or any further nutrients as described herein for the inventive composition, particularly LC-PUFA and iron, as both described herein.

Preferably, the composition of the present invention, preferably obtained or obtainable by a method as described herein for preparing a composition, may be used for prevention, amelioration or treatment of a disease or disorder as defined herein. As used herein, the term “a disorder” or “a disease” refers to any derangement or abnormality of function; a morbid physical or mental state. See Dorland's Illustrated Medical Dictionary, (W.B. Saunders Co. 27th ed. 1988). Such diseases or disorders may be selected from malnutrition, metabolic diseases, neurodegenerative diseases, Alzheimer disease/cognitive impairment, Parkinson's disease, neurological diseases, Amyotrophic lateral sclerosis, Traumatic brain injury, Hypoxic/ischemic brain injury, Autism, ADHD (Attention Deficit Hyperactivity Disorder), Depression, Headaches, Migraine Headaches, Narcolepsy, GLUT-1 deficiency, Pyruvate Dehydrogenase (PDH) deficiency, phosphofructokinase (PFK) deficiency, Glycogenosis type V (McArdle disease), Cardiac ischemia, Rett syndrome, Tuberous Sclerosis, Diabetes and Cancer (astrocytornas, prostate, gastric, renal, head and neck), preferably for use in the prevention, amelioration or treatment of malnutrition, metabolic diseases, neurodegenerative diseases, preferably as a nutritional supplement. The composition is preferably used as a nutritional composition or supplement.

The composition of the present invention may also be used for the promotion of the development of the nervous system and/or of the retina, and/or in the promotion and/or improvement of the mental performance, behavioural and visual functions of an infant or a child.

For the purpose of the present invention, mental performance is for example intended as cognitive and intellectual performance, memory, as well as language ability of an infant or child. Development of the nervous system is intended to include for example brain and neuronal development.

The composition of the present invention may further be used to strengthen immunity, including the development of gut microflora.

The composition of the present invention furthermore can be used for reducing the risk of the development of overweight, obesity and insulin resistance.

The advantageous effects of the inventive composition as described above is preferably accomplished by administering an effective amount of a composition according to the present invention to a subject in need thereof. Preferably, such a composition is to be administered once daily, preferably twice daily, more preferably three times daily, wherein during administration preferably at least one unit or dose for administration is provided, as defined herein. Upon administration, preferably the total amount of energy to be administered per day is as defined before. As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human, more preferably selected from an infant, a child or an adult. The term “effective amount” of a composition of the present invention refers to an amount of the compound of the present invention that will elicit the biological or medical response of a subject, enhance development or organs or functions of a subject, or ameliorate symptoms, slow or delay disease progression, or prevent a disease, etc. Preferably, such an “effective amount” is a packaged dose or unit as obtained as described herein.

Particularly preferably, the inventive compositions as described herein, either as described initially or as obtained or obtainable according to the inventive process, are preferably suitable for use in infants, e.g. for nutrition, supplemental nutrition, or treatment of a disease as defined herein. The present invention is however also suitable for use by adults and children, preferably for nutrition, supplemental nutrition, or treatment of a disease as defined herein.

The present invention also includes a method of treatment by administering a patient in need thereof a composition as defined herein for a disease or disorder as defined herein.

Most preferably in the compositions utilized in the inventive method of treatment of the present invention the composition is a solid, a liquid or a semi liquid/semi solid, even more preferably is a liquid.

In said inventive method of treatment the inventive composition is preferably in the form of a supplement. It is alternatively preferable that in said method of treatment the inventive composition is used as a sole source of nutrition as defined herein.

It is particularly preferable in the inventive method of treatment of the present invention that the composition, which is preferably a solid, semi-liquid/semi-solid composition, most preferably a liquid composition is adjusted to neutral or acidic pH, preferably a pH comprised between about 3.5 and about 7.5, preferably between about 4 to about 7, or according to the ranges described herein above.

Various embodiments of the invention have been described above. The descriptions are intended to be illustrative, not limitative. Thus, it will be apparent to one skilled in the art that certain modifications maybe made to the invention as described without departing from the scope of the claims set out below.

Unless otherwise indicated, the term “at least” in the context of the present invention typically preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

For example, as described herein, “preferred embodiment” means “preferred embodiment of the present invention”. Likewise, as described herein, “various embodiments”and “another embodiment” means “various embodiments of the present invention” and “another embodiment of the present invention”, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”. When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Furthermore, percentages as described in the present invention can be interchangeably either % weight-by-weight (w/w) or % weight-by-volume (w/v), if not specifically indicated otherwise.

Finally, all publications and patents cited in this disclosure are incorporated by reference in their entirety. To the extent the material incorporated by reference contradicts or is inconsistent with this specification, the specification will supersede any such material.

### EXAMPLES

The following examples are intended to illustrate the invention further. They are not intended to limit the subject matter of the invention thereto.

### Example 1

Test emulsions were prepared at pH 7 and at pH 4. At both pH, emulsions were prepared combining the forms of fish oil (free or encapsulated) mentioned in the tables below with
each of the forms of iron mentioned in the tables below,
each of the proteins mentioned in the tables below and
each of the antioxidants mentioned in the tables below,
   in the presence or absence of medium chain triglycerides (MCT).

Tables 1 and 2 below list the ingredients used and corresponding amount in the test emulsions.

TABLE 1
design of experiments at pH 7

| | Parameters | | | | |
|---|---|---|---|---|---|
| | | | | | Anti- |
| | Fish Oil | Iron | Proteins | Other oil | oxidant |
| Ingre- | NIF oil | Ferrous | Caseinate | Nothing | Vitamin C |
| dients | (Sofinol)1) | sulfate | | | |
| | | 7 hydrate | | | |
| | Concentrated | Ferric | Whey | MCT oil | Vitamin E |
| | fish oil | pyrophos- | | | |
| | (BASF)5) | phate | | | |
| | NIF | Ferric | | | |
| | powder 7 | citrate | | | |
| | (Kievit)2) | | | | |
| | Intelligentia | Ferrous | | | |
| | (GAT) 3) | bis- | | | |
| | | glycinate | | | |
| | | (chelate) | | | |
| | | Ferric | | | |
| | | saccharate | | | |
| | | encapsu- | | | |
| | | lated | | | |
| | | Nothing | | | |
| Level/ | 150 mg | 5 mg/100 g | 1 g/100 g | 2 g/100 g | 20 mg/100 |
| 100 g | DHA/100 g | | | | g for C |
| | | | | | 4 mg/100 |
| | | | | | g for E |

1)pure fish oil containing around 30% of DHA & EPA
2)LC-PUFA microencapsulated in a glassy matrix of dairy proteins and glucose
3) encapsulated in an alginate matrix (AB-Fortis)
5)concentrated fish oil containing 60% of DHA & EPA

TABLE 2
design of experiments at pH 4

| | Parameters | | | | |
|---|---|---|---|---|---|
| | | | | | Anti- |
| | Fish Oil | Iron | Proteins | oil | oxidant |
| Ingre- | NIF oil | Ferrous | Nothing | Nothing | Vitamin C |
| dients | (Sofinol)1) | sulfate | | | |
| | | 7 hydrate | | | |
| | Concentrated | Ferrous | Whey | MCT | Vitamin E |
| | fish oil | bis- | | | |
| | (BASF)5) | glycinate | | | |
| | NIF | Ferric | | | |
| | powder 7 | pyrophos- | | | |
| | (Kievit)2) | phate | | | |
| | Powder Loc | Ferric | | | |
| | (ONC- | saccharate | | | |
| | DSM)4) | encapsu- | | | |
| | | lated4) | | | |
| Level/ | 150 mg | 5 mg/100 g | 1 g/100 g | 2 g/100 g | 20 mg/100 |
| 100 g | DHA/100 g | | | | g for C |
| | | | | | 4 mg/100 |
| | | | | | g for E |

1)pure fish oil containing around 30% of DHA & EPA
2)LC-PUFA microencapsulated in a glassy matrix of dairy proteins and glucose
3) encapsulated in an alginate matrix (AB-Fortis)
4)Emulsion with double layer of cross-linked gelatin
5)concentrated fish oil containing 60% of DHA & EPA

Emulsions were prepared using the following method. The fish oil, if present the MCT, an emulsifier and if present vitamin E were mixed at room temperature. Then, this mixture was dispersed in water and the protein, if present the vitamin C, and iron were added. The resulting emulsion was homogenized with an ultraturrax at 12000 U/min during 6 min. The pH was then adjusted to pH 7 or pH 4 by addition of citric acid. The emulsion was then homogenized at 400 and 600 bars on a mini-homogenizer (representing lower pressure values on a bench scale homogenizer). Then, the emulsions underwent a heat treatment (80° C.-30 sec). They were then bottled in aseptic plastic containers.

Each emulsion was then subjected to a sensory evaluation by a trained professional panel (10-12 persons). The panelists tasted the emulsions one day after their preparation. The fishy odor, fishy flavor, painty odor and painty flavor were evaluated on a scale ranging from 0 to 5 (5 being the most fishy/painty intensity and 0 being no odor or flavor). The tasting session was done in individual booth, at ambient temperature. The contribution of each of the ingredients to the fishy flavor and odor and painty flavor and odor was then assessed using statistic methods.

As the sensory data for “fishy odor” was giving the exact same tendency than for “fishy flavor” and as sensory data for the “painty odor” was giving the exact same tendency as for “painty flavor”, we chose to explain in this part only “fishy flavor” and “painty flavor” results.

Microencapsulated forms of fish oil were shown to have reduced off-flavor compared to free oil. The fish oil performing the best in terms of fishy and painty off-notes is NIF powder 7 at both at pH 7 and 4. The iron form performing the best in term of fishy and painty off-notes at pH7 is the ferric pyrophosphate and the ferric saccharate encapsulated in a calcium alginate matrix (from AB Fortis). These two iron forms lead to the same intensity of off-notes than the variant without iron. At pH4, only the ferric saccharate encapsulated in a calcium alginate matrix had an impact for reducing fishy and painty off-notes. The combination of microencapsulated fish oil (most particularly NIF powder 7) and of ferric saccharate gave the best results in terms of off-flavor reduction both for fishy and painty flavor.

At pH 7, the protein performing the best in term of fishy and painty off-notes was the whey protein. The sensory score in term of paintiness and fishiness were better when Medium Chain Triglyceride (MCT) was present, at pH7 and 4.

The use of antioxidant proved to be efficient to further reduce the painty and fishy off-notes. The antioxidant performing the best in term of fishy and painty off-notes in the emulsions was tocopherol (vitamin E) both at pH7 and pH4.

1. A composition fortified with ferric saccharate and a high concentration of microencapsulated LC-PUFA.

2. The composition according to claim 1 wherein the LC-PUFA is present in about 0.02 to 10% by weight of the composition.

3. The composition according to claim 1, wherein the LC-PUFA is microencapsulated in a glassy matrix of dairy proteins and glucose, the LC-PUFA preferably being derived from fish oil or algae oil.

4. The composition according to claim 1 wherein ferric saccharate provides an amount of iron of about 0.0001% to about 1% by weight of the composition.

5. The composition according to claim 1, wherein the ferric saccharate is microencapsulated.

6. The composition according to claim 1, which contains at least one antioxidant which is a radical scavenger.

7. The composition according to claim 1, which contains a non-sensitive fat.

8. The composition according to claim 1, which is in a form selected from the group consisting of a food matrix, a beverage and a food supplement.

9. The composition according to claim 8, wherein the food product is a nutritional composition.

10. The composition according to claim 1 which is a pharmaceutical composition and/or a nutraceutical product.

11. A method for use in the prevention, amelioration or treatment of malnutrition, metabolic diseases, neurodegenerative diseases comprising administering to an individual in need of same a composition comprising a composition fortified with ferric saccharate and a high concentration of microencapsulated LC-PUFA.

12. A method for use in the promotion of the development of the nervous system and/or of the retina, in the promotion and/or improvement of the mental performance, behavioural and visual functions of an infant or a child, for strengthening immunity, including the development of gut microflora, and/or for reducing the risk of the development of overweight, obesity and insulin resistance comprising administering to an individual in need of same a composition comprising a composition fortified with ferric saccharate and a high concentration of microencapsulated LC-PUFA.

13. A method for preventing or reducing the oxidation of LC-PUFA in a composition comprising LC-PUFA and iron, comprising adding LC-PUFA to the composition in microencapsulated form and adding iron in the form of ferric saccharate.

14. A method for preventing or reducing the off-taste of LC-PUFA in a composition comprising LC-PUFA and iron, comprising adding LC-PUFA to the composition in microencapsulated form and adding iron in the form of ferric saccharate, wherein preferably the LC-PUFA is microencapsulated in a glassy matrix of dairy proteins and glucose.

15-16. (canceled)
